(19) United States
(12) Patent Application Publication
(10) Pub. No.: US 2012/0325219 A1
(43) Pub. Date: Dec. 27, 2012

(54) PATIENT INTERFACE DEVICE WITH TUBING ASSEMBLY

(75) Inventor: David W. Smith, Oakmont, PA (US)

(73) Assignee: KONINKLIJKE PHILIPS ELECTRONICS N.V., EINDHOVEN (NL)

(21) Appl. No.: 13/583,077

(22) PCT Filed: Feb. 14, 2011
(86) PCT No.: PCT/IB11/50610

§ 371 Date: Sep. 6, 2012

Related U.S. Application Data
(60) Provisional application No. 61/311,431, filed on Mar. 8, 2010.

(51) Int. Cl. A61M 16/06

(52) U.S. Cl. 128/205.25

(57) ABSTRACT

A patient interface device (10) that includes a patient sealing element (12) and a tubing assembly (14) fluidly coupled to the patient sealing element for delivering a breathing gas to the patient sealing element. The tubing assembly is adapted to be worn on a head of a user and includes at least one rigid or semi-rigid straight segment (20) fluidly coupled to at least one flexible bellows segment (18). The tubing assembly may be provided within a headgear component (16) used to attach the patient interface device to the patient's head.

This patent application claims the priority benefit under 35 U.S.C. §119(e) of U.S. Provisional Application No. 61/311,431 filed on Mar. 8, 2010, the contents of which are herein incorporated by reference.

The present invention relates to patient interface devices for transporting a gas to and/or from an airway of a user, and, in particular, to a patient interface device that include straight and flexible bellows segments that may, for example, be provided within a headgear component used to attach the patient interface device to the patient's head.

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head. Because such patient interface devices are typically worn for an extended period of time, it is important for the headgear to maintain the mask component of the device in a tight enough seal against the patient's face without discomfort.

A number of known patient interface devices provide airflow to the patient through the headgear via one or more delivery conduits that warp around portions of the head as part of the headgear. Such known patient interface devices, however, have a number of drawbacks. For example, such known patient interface devices do not readily allow for size adjustment of the tubing, effectively balance patient comfort with tubing rigidity, provide for simplified cleaning of the tubing, and/or effectively manage noise in proximity to the wearer's ears.

Accordingly, it is an object of the present invention to provide a patient interface device that overcomes the shortcomings of conventional patient interface devices. This object is achieved according to one embodiment of the present invention by providing a patient interface device that includes a patient sealing element and a tubing assembly fluidly coupled to the patient sealing element for delivering a breathing gas to the patient sealing element. The tubing assembly includes at least one substantially rigid straight segment fluidly coupled to at least one flexible bellows segment. In one exemplary embodiment, the tubing assembly includes a plurality of straight segments and a plurality of bellows segments.

In a further embodiment, the tubing assembly includes a first arm structured to rest along first side a head of a patient and a second arm structured to rest along second side a head of a patient when the patient interface device is donned by the patient. A first end of the first arm and a first end of the second arm are each fluidly coupled to the patient sealing element, and a second end of the first arm and a second end of the second arm are each fluidly coupled to a coupling connector. In addition, the patient interface device may also include headgear component for securing the patient interface device to the head of the patient, the headgear component having a first side sleeve encasing the first arm and a second side sleeve encasing the second arm.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

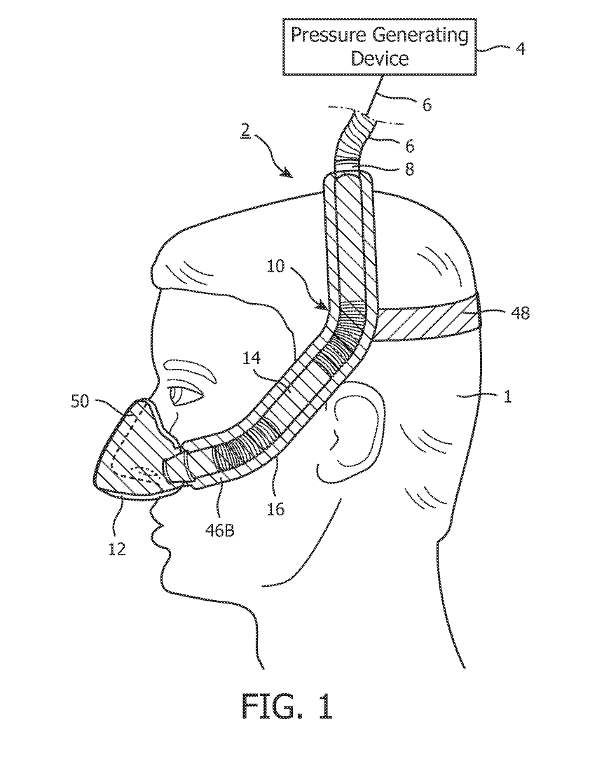

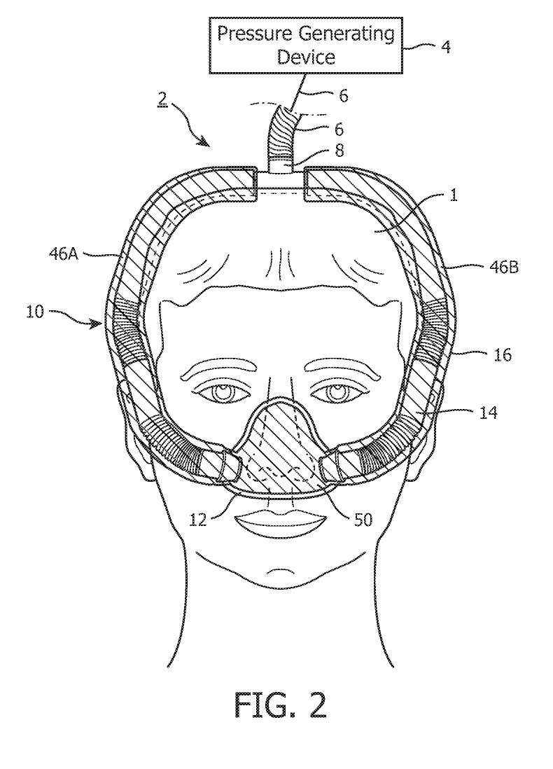

FIGS. 1 and 2 are side and front schematic diagrams, respectively, of a system adapted to provide a regimen of respiratory therapy to a patient according to one exemplary embodiment of the present invention;

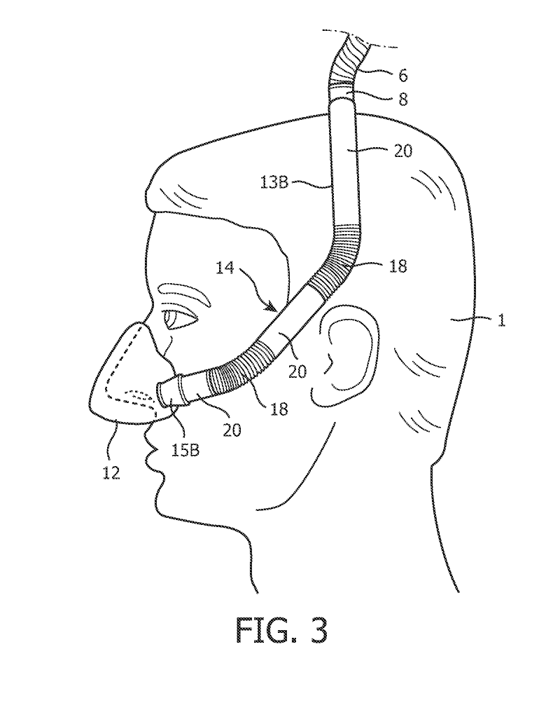

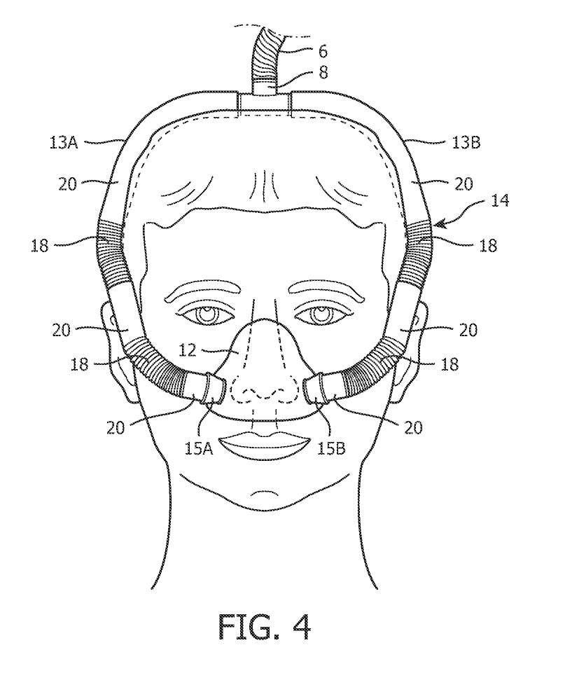

FIGS. 3 and 4 are partial side and front schematic diagrams, respectively, of the system of FIGS. 1 and 2 wherein the headgear component of the patient interface device of the system has been removed in order to more readily show the patient sealing element and tubing assembly of the patient interface device;

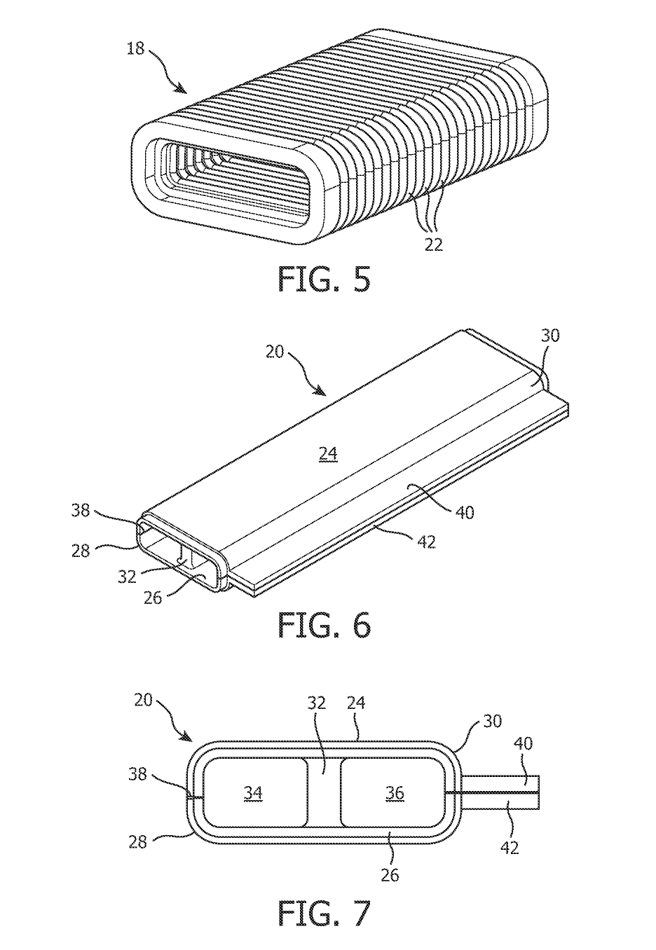

FIG. 5 is an isometric view of a bellows segment according to an exemplary embodiment forming part of the tubing assembly shown in FIGS. 1-4;

FIGS. 6 and 7 are isometric and front views, respectively, of a straight segment according to an exemplary embodiment forming part of the tubing assembly shown in FIGS. 1-4;

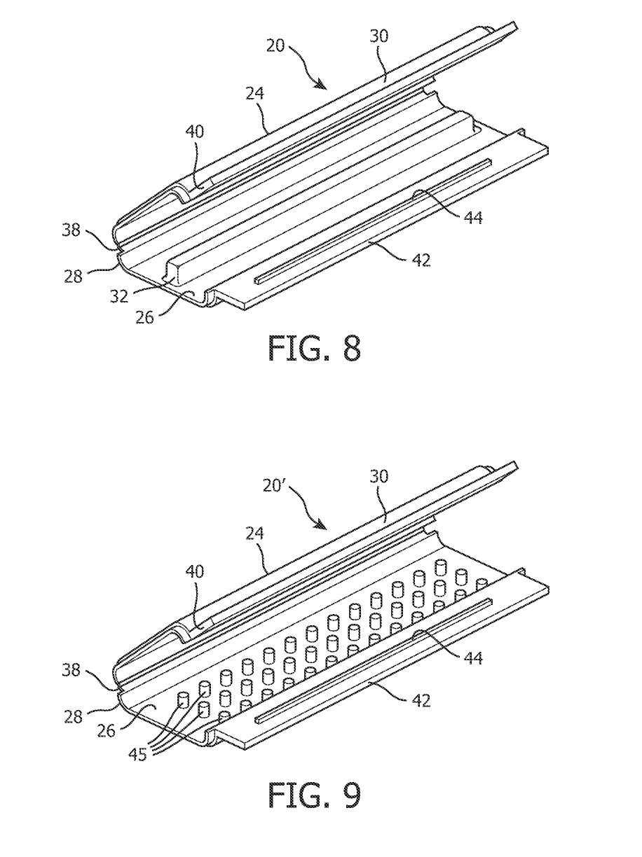

FIG. 8 is an isometric view of the straight segment of FIGS. 6 and 7 in an open condition which facilitates cleaning of the straight segment; and

FIG. 9 is an isometric view of a straight segment 20 according to an alternative exemplary embodiment.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIGS. 1 and 2 are side and front schematic diagrams, respectively, of a system 2 adapted to provide a regimen of respiratory therapy to a patient 1 according to one exemplary embodiment of the present invention. System 2 includes a pressure generating device 4, a delivery conduit 6 fluidly coupled to a coupling connector 8, and a patient interface device 10 fluidly coupled to coupling connector 8. Pressure generating device 4 is structured to generate a flow of positive pressure breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 10 through coupling connector 8 (the breathing gas enters at the top of the head of patient 1). Delivery conduit 6, coupling connector 8 and patient interface device 10 are often collectively referred to as a patient circuit.

As described in greater detail herein, patient interface device 10 includes a patient sealing element 12, a tubing assembly 14 that is fluidly coupled to both coupling connector 8 and patient sealing element 12. Headgear component 16 structured to receive and hold tubing assembly 14 and maintain patient interface device 10 on the face/head of patient 1. FIGS. 3 and 4 are partial side and front schematic diagrams, respectively, of system 2 wherein headgear component 16 has been removed in order to more readily show patient sealing element 12 and tubing assembly 14. In the exemplary embodiment, patient sealing element 12 is a nasal cushion made of a soft, flexible material, such as, without limitation, silicone, an appropriately soft thermoplastic elastomer, a closed cell foam, or any combination of such materials. However, any type of patient sealing element, such as a nasal/oral mask, a nasal pillow or a full face mask, which facilitates the delivery of the flow of breathing gas to the airway of a patient, may be used as mask component 12 while remaining within the scope of the present invention.

Referring to FIGS. 3 and 4, tubing assembly 14 includes left and right side arms 13A, 13B (fluidly coupled to a respective side of coupling component 8), each made up of a number of individual tubing segments wherein the tubing segments include a plurality of flexible bellows segments 18 and a plurality of straight segments 20. The present invention contemplates that straight segments 20 include a soft, flexible material, such as, without limitation, silicone, an appropriately soft thermoplastic elastomer, a closed cell foam, or any combination of such materials. The present invention further contemplates that segments 20 can also be made from a rigid or semi-rigid material, such as a 50-60 Shore A durometer silicone. These segments can also be made from a combination of materials, such as a semi-rigid silicon covered with a soft covering material.

Each side arm 13A, 13B also includes a respective coupling component 15A, 15B that is fluidly coupled to the other segments of the associated side arm 13A, 13B at one end thereof and is fluidly coupled to the interior of patient sealing element 12 at the other end thereof. As seen in FIGS. 3 and 4, bellows segments 18, straight segments 20 and coupling components 15A, 15B are fluidly interconnected (e.g., by a friction fit between each component) to form tubing assembly 14 for delivering a flow of gas generated by pressure generating device 4 to patient sealing element 12 and ultimately to the airway of patient 1. In the illustrated embodiment, the breathing gas thus flows through both left side arm 13A and right side arm 13B to patient sealing element 12, thereby minimizing resistance to flow. In addition, as described in greater detail elsewhere herein, the interconnection of bellows segments 18 and straight segments 20 allows the length and shape of tubing assembly 14 to be readily adjusted in order to provide a good fit for patient 1.

Furthermore, while in the illustrated embodiment each side arm 13A, 13B includes two bellows segments 18 and two straight segments 20, it should be understood that this is meant to be exemplary only and that more or less bellows segments 18 and straight segments 20 may be used in each side arm 13A, 13B as dictated by the needs of the particular application. In addition, the length of each bellows segment 18 and/or straight segment 20 may be varied as needed to suit the particular application.

FIG. 5 is an isometric view of bellows segment 18 according to an exemplary embodiment. As seen in FIG. 5, bellows segment 18 is an elongated, hollow member having an oblong (e.g., rounded rectangular) cross-section. In addition, bellows segment 18 has a convolution geometry that includes a number of pleated sections 22 having vertical walls that allow bellows segment 18 to bend and elongate when forces are applied thereto. In the exemplary embodiment, the convolution geometry has an aspect ratio, defined as convolution height to width, in the range of approximately 2:1 to 1:3, and a wall thickness in the range of approximately 0.2 mm to 0.5 mm that together allow bellows segment 18 to be elongated to up to approximately three times its original length. In addition, each bellows segment 18 is, in the exemplary embodiment, molded from a stiff yet flexible material, such as plastic or silicone, that permits the bending and elongation described herein. The bending and elongation as just described allows for selective adjustment of tubing assembly 14 to achieve a proper fit without braking the airflow seal (within tubing assembly 14 and between patient sealing element 12 and the face of patient 1).

FIGS. 6 and 7 are isometric and front views, respectively, of straight segment 20 according to an exemplary embodiment. FIG. 8 is an isometric view of straight segment 20 in an open condition, which, as described elsewhere herein, facilitates cleaning of straight segment 20. As seen in FIGS. 6-8, straight segment 20 is, like bellows segment 18, an elongated, hollow member having an oblong cross-section. Straight segment 20 includes top wall 24, bottom wall 26, left side wall 28 and right side wall 30. In addition, straight section 20 further includes a central rib 32 that provides structural support for straight member 20 to prevent it from collapsing under pressure (e.g., pressure that may result from a patient's head resting on a pillow). In addition, in the illustrated embodiment, each bellows section 18 is surrounded (bounded at each end) by a pair of straight sections 20. This reduces the chances that a bellows section 18 will be crushed or collapsed (causing airflow occlusion) during use.

Central rib 32 also defines first chamber 34 and second chamber 36 within straight section 20. The provision of the two chambers 34, 36 optimizes the cross-sectional area of the passageway through straight section 20 (i.e., the surface area to cross section ratio is minimized) while providing for minimum airflow resistance. One advantage of keeping this ratio low is that it allows for relatively small overall tube size in tubing assembly 14, which allows tubing assembly 14 to be shaped to the face of patient 1 for optimum comfort. In one exemplary embodiment, the height of straight section 20 is between approximately 3 mm and approximately 6 mm and the width of straight section 20 (ignoring central rib 32) is between approximately 12 mm and approximately 25 mm. This is in contrast to common, prior art headgear airflow systems which employ several parallel sections of small diameter tubing, resulting in a relatively high surface area to cross section ratio and therefore restricted airflow. In the exemplary embodiment, straight section 20 is molded from a stiff yet flexible material, such as plastic or silicone. Rather than being semi-rigid, the present invention also contemplates that the straight sections can be rigid, i.e., substantially not flexible.

In addition, as shown in FIG. 8, straight section 20 according to the illustrated embodiment may be opened to facilitate the cleaning of the inside of straight section 20. In particular, left side wall 28 is provided with a living hinge 38 that allows top wall 24 to be pivoted way from bottom wall 26 to provide access to the interior of straight section. In addition, a locking mechanism 44, such as, without limitation, a tongue and groove arrangement, may be provided on flanges 40, 42 that are attached to right side wall 30 in order to maintain straight section 20 in a closed condition when in use.

FIG. 9 is an isometric view of straight segment 20′ according to an alternative exemplary embodiment. Straight segment 20′ is similar to straight segment 20, and like components are labeled with like reference numerals. However, rather than including central rib 32, straight segment 20′ includes a plurality of cylindrical support columns 45 extending upwardly from bottom wall 26 which, like central rib 32, provide structural support for straight member 20′ to prevent it from collapsing under pressure.

Referring again to FIGS. 1 and 2, in the exemplary embodiment, tubing assembly 14 is at least partially incased within headgear component 16. As seen in FIGS. 1 and 2, headgear component 16 includes left side sleeve 46A which receives and holds therein (i.e. encases) left side arm 13A and right side sleeve 46B which receives and holds therein (i.e. encases) left side arm 13B. In addition, rear strap 48 is provided between side sleeves 46A, 46B, and is structured to engage the rear of the head of patient 1. Patient sealing element cover 50 is provided between the ends of side sleeves 46A, 46B, and is structured to engage patient sealing element 12 and hold it in place against the face of patient 1 in order to provide a force sufficient to cause an appropriate seal between patient sealing element 12 and the face of patient 1. A number of well known mechanism for adjusting the size and fit of headgear component 16, such as adjustable straps, may be provided as part of headgear component 16. Because such mechanism are well known in the art, they will not be described in detail herein.

In the exemplary embodiment, at least part of headgear component 16 is made of a material that will dampen noise from airflow through tubing assembly 14, in particular in the region near the ears of patient 1. For example, side sleeves 46A and 46B may be made of such a noise dampening material. Headgear component 16 may be made of a single layer of, or a combination of layers of, one or more of the following noise dampening materials: fabric, foam, viscoelastic foam, molded silicone, and a gel material. In one particular embodiment, headgear component 16 is made form a combination of fabric and foam materials.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a patient sealing element; and
a tubing assembly fluidly coupled to the patient sealing element and adapted to be worn on a head of a user for delivering a breathing gas to the patient sealing element, the tubing assembly including a rigid or semi-rigid first straight segment fluidly coupled to a flexible first bellows segment comprising a plurality of pleated sections, wherein the pleated sections each have a convolution geometry having an aspect ratio defined as convolution height to convolution width in the range of approximately 2:1 to 1:3 and a wall thickness of approximately 0.2 mm to 0.5 mm such that the first bellows segment is able to be elongated up to three times its original width.

2. The patient interface device according to claim 1, wherein the tubing assembly includes a second straight segment and a second bellows segment comprising a plurality of second pleated sections, wherein the second pleated sections each have a second convolution geometry having an aspect ratio defined as convolution height to convolution width in the range of approximately 2:1 to 1:3 and a wall thickness of approximately 0.2 mm to 0.5 mm such that the second bellows segment is able to be elongated up to three times in its original width.

3. The patient interface device according to claim 2, wherein the tubing assembly includes a first arm structured to rest along a first side a head of a patient and a second arm structured to rest along a second side a head of a patient when the patient interface device is donned by the patient, wherein a first end of the first arm and a first end of the second arm are each fluidly coupled to the patient sealing element, and wherein a second end of the first arm and a second end of the second arm are each fluidly coupled to a coupling connector.

4. The patient interface device according to claim 3, wherein the first arm includes the first straight segment and the first bellows segment in fluid communication with one another, and the second arm includes the second straight segment and the second bellows segment in fluid communication with one another.

5. The patient interface device according to claim 4, wherein the coupling connector is structured to rest on top of the head of the patient when the patient interface device is donned by the patient.

6. The patient interface device according to claim 5, further comprising a headgear component for securing the patient interface device to the head of the patient, the headgear component having a first side sleeve encasing the first arm and a second side sleeve encasing the second arm.

7. The patient interface device according to claim 6, wherein the first and second side sleeves are made from one or more noise dampening materials.

8. The patient interface device according to claim 6, wherein the headgear component includes a patient sealing element cover covering the patient sealing element, the patient sealing element cover being coupled to the first side sleeve and the second side sleeve.

9. The patient interface device according to claim 2, wherein the first straight segment and the second straight segment are each selectively openable.

10. The patient interface device according to claim 9, wherein the first straight segment includes a first top wall and a first bottom wall, wherein the first top wall is selectively pivotable relative to the first bottom wall on a first hinge, and wherein the second straight segment includes a second top wall and a second bottom wall, wherein the second top wall is selectively pivotable relative to the second bottom wall on a second hinge.

11. The patient interface device according to claim 10, wherein the first top wall has a first top flange, the first bottom wall has a first bottom flange, wherein the first top flange and the first bottom flange comprise a first locking mechanism for selectively locking the first straight segment in a closed condition, and wherein the second top wall has a second top flange, the second bottom wall has a second bottom flange, wherein the second top flange and the second bottom flange comprise a second locking mechanism for selectively locking the second straight segment in a closed condition.

12. (canceled)

13. The patient interface device according to claim 1, wherein the first straight segment includes a central support rib extending along a longitudinal axis thereof, the central rib defining a first chamber and a second chamber within the interior of the first straight segment.

14. The patient interface device according to claim 1, wherein the first straight segment includes a plurality or cylindrically-shaped support columns extending outwardly from either a top wall or bottom wall thereof.

15. The patient interface device according to claim 7, wherein the first and second side sleeves are made from molded silicone and/or a gel material.
